(19) United States
(12) Patent Application Publication
(10) Pub. No.: US 2006/0085067 A1
(43) Pub. Date: Apr. 20, 2006

(54) Intervertebral implant

(76) Inventors: Thomas Gradel, Ayze (FR); Jean-Philippe Lemaire, Saulon La Chapelle (FR)

Correspondence Address: WILLIAM H. EILBERG, THREE BALA PLAZA, SUITE 501 WEST, BALA CYNWYD, PA, 19004, US

(21) Appl. No.: 10/539,160

(22) PCT Filed: Dec. 16, 2003
(86) PCT No.: PCT/FR03/03734

§ 371 Date: Jun. 14, 2005

(30) Foreign Application Priority Data
Dec. 17, 2002 (FR) 02/16234

(51) Int. Cl. A61F 2/44

(52) U.S. Cl. 623/17.11

(57) ABSTRACT

An intervertebral implant includes an essentially tubular body which is made from a biocompatible solid material. The body includes a large inner cavity which is defined by upper and lower walls, lateral walls, a front wall, and a rear wall, the rear wall having a tapped axial hole. A replaceable compression plug, which can be adapted by screwing, is inserted into the hole, in order to compress a graft which is disposed in the inner cavity of the implant.

### TECHNICAL FIELD OF THE INVENTION

The present invention relates to intervertebral implants for stabilization of the vertebral column adapted to form a wedge to be inserted between the facing plates of two adjacent vertebrae to maintain a constant disc spacing.

The document EP 0 307 241 A describes one example of an intervertebral implant taking the form of a cage of parallelepiped shape. This form of cage is not well suited to use in the vertebral column, in particular because the longitudinal edges of this kind of cage may cause lesions to the spinal cord or the roots during insertion of the implant between the adjacent vertebrae. Relatively ineffective retention of the adjacent vertebrae is also encountered, and is likely to be caused by the parallelepiped shape of the cage.

There has also been proposed an intervertebral implant of parallelepiped shape in which the upper and lower larger faces are plane and at an angle to each other that is open toward the front, to maintain the adjacent vertebrae at an appropriate physiological lordosis angle. The shape of the implant necessitates the formation of large recesses in the vertebral plates, to allow insertion of the intervertebral implant by front to back movement in translation during fitting, which creates inappropriate trauma.

The document U.S. Pat. No. 5,888,227 A describes an intervertebral implant with a peripheral wall around a single large interior cavity open at the front and at the rear. The upper and lower faces are divergent toward the front and have a double front-to-back and transverse curvature. The structure has insufficient mechanical strength and the retention of the adjacent vertebrae is insufficient.

The document WO 02/15825 A describes an implant of cylindrical general shape with upper and lower walls each provided with two orifices, an anterior wall with a threaded hole for manipulation by a fitting tool, and lateral walls each having a longitudinal groove whose edges oppose axial rotation of the implant during fitting.

### STATEMENT OF INVENTION

The problem addressed by the present invention is that of defining a new intervertebral implant structure that very significantly reduces the risks of nervous lesions during fitting, improves positioning and locking of the vertebrae to each other, and minimizes bone trauma during fitting. Particular requirements are improved mechanical strength and good bone attachment, facilitating fusion of two adjacent vertebrae.

To achieve the above and other objects, the intervertebral implant of the invention for stabilizing adjacent vertebrae, comprises a solid biocompatible material implant body having a tubular general structure delimited by an upper wall and a lower wall that are convex and slightly divergent toward the front, two opposite lateral walls that are plane and slightly divergent toward the front, and a posterior wall with a threaded axial hole, with a single interior cavity providing communication between orifices provided in the upper wall and the lower wall; according to the invention:

the upper wall and the lower wall each comprise a respective single large upper orifice or lower orifice,

an interchangeable compression plug is adapted to be fitted by screwing it into the threaded axial hole in the posterior wall,

the interior cavity is closed toward the front by an anterior wall,

the width of the implant defined by the lateral walls is less than its height defined by the upper wall and the lower wall.

A structure of the above kind with a large central cavity and large openings in the upper and lower walls encourages contact between a graft inserted inside the implant body and the two vertebral plates between which the intervertebral implant is inserted. Fitting the graft is facilitated. The interchangeable compression plug compresses the graft against the two adjacent vertebrae after fitting. Finally, the implant may be fitted between two vertebrae by being turned 90° so that its lateral faces that are closer together are at the top and at the bottom, facing the vertebral plates of the two adjacent vertebrae. It is then rotated 90° to bring the graft into contact with the vertebrae. This reduces the trauma to the vertebral region and minimizes the quantity of material that has to be removed to insert the intervertebral implant between the adjacent vertebrae.

In one advantageous embodiment, because of the upper orifice and the lower orifice, the interior cavity is open over the whole of its width between the lateral walls and over the whole of its length between the posterior wall and the anterior wall. This facilitates fitting the graft and maximizes its contact with the adjacent vertebrae.

The interchangeable compression plug preferably comprises a conical interior end portion. This facilitates penetration of the plug into the graft, to compress it.

To obtain effective compression the interchangeable compression plug and the threaded axial hole that receives it advantageously have a diameter substantially equal to the width of the interior cavity in the vicinity of the posterior wall.

Good results are obtained if the interchangeable compression plug has a length such that, at the end of screwing it into the threaded axial hole that receives it, its interior end portion penetrates the interior cavity to a distance of at least one quarter of the length of said interior cavity.

The implant is preferably provided with at least two interchangeable compression plugs having different lengths, so that the practitioner can choose the most suitable plug for compressing the graft.

In one advantageous embodiment, the posterior wall of the implant body includes an external diametral groove for actuating axial rotation of the implant, to move it from an insertion position, with its lateral walls at the top and at the bottom, to a vertebrae supporting orientation with the upper and lower walls bearing on the adjacent vertebral plates.

The implant may be made from a solid biocompatible material such as titanium or advantageously from a PEEK type polymer, which has the advantage of being radio transparent.

### BRIEF DESCRIPTION OF THE DRAWINGS

Other objects, features and advantages of the present invention will emerge from the following description of particular embodiments given with reference to the appended drawings, in which:

FIG. 1 is a ¾ rear perspective view of an intervertebral implant body of one embodiment of the present invention;

FIG. 2 is a ¾ front perspective view of the FIG. 1 implant body;

FIGS. 3 to 6 show the structure of the implant body from FIGS. 1 and 2 from above, from the side, from the front, and from the rear, respectively;

FIG. 7 is a ¾ rear perspective view of an implant of the invention comprising the implant body from FIG. 1 and an interchangeable compression plug; and

FIG. 8 is a plan view of the FIG. 7 implant.

### DESCRIPTION OF PREFERRED EMBODIMENTS

In the embodiment shown in the figures, the intervertebral implant of the invention comprises a solid biocompatible material implant body 1 that has a tubular general structure delimited by an upper larger wall 2 and a lower larger wall 3 that are convex and slightly divergent toward the front, two opposite lateral walls 4 and 5 that are plane and slightly divergent toward the front, a posterior wall 6 with a threaded axial hole 7, and an anterior wall 8.

The interior of the implant body 1 is a single interior cavity 9, open at the top via an upper orifice 10 and open at the bottom via a lower orifice 11. The interior cavity 9 therefore provides communication between the upper orifice 10 and the lower orifice 11 provided in the upper larger face 2 and the lower larger face 3 of the implant body 1.

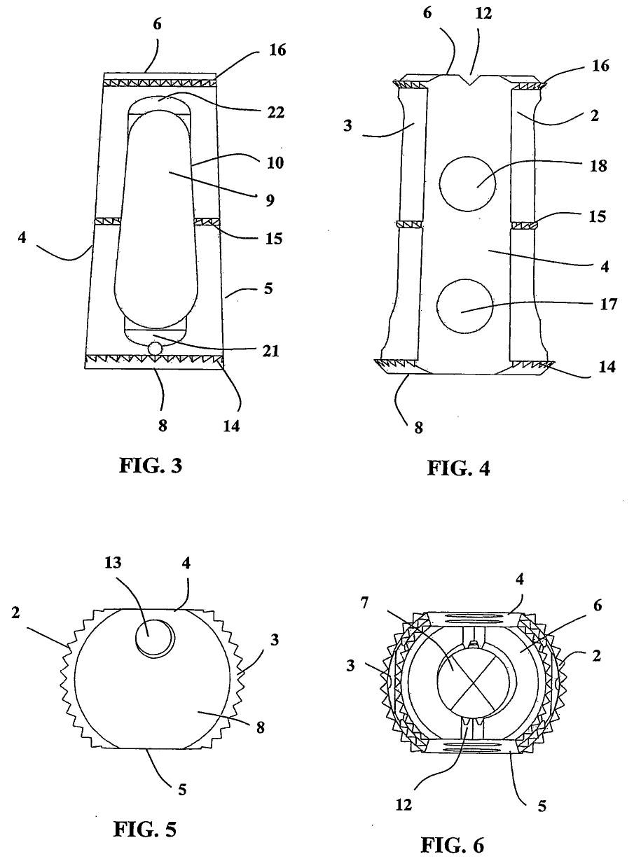

In the embodiment shown in the figures, as seen best in FIG. 3, the interior cavity 9 has the general shape of a truncated pyramid that widens in the direction of the anterior wall 8. The upper orifice 10 and the lower orifice 11 mean that the interior cavity 9 is open over the whole of its width between the lateral walls 4 and 5 and over the whole of its length between the posterior wall 6 and the anterior wall 8.

The posterior wall 6 has an external diametral groove 12 for engaging a tool such as a screwdriver blade for imparting axial rotation to the implant body.

The anterior wall 8 includes an eccentric threaded hole 13, as shown in FIG. 2 or FIG. 5, the eccentric threaded hole 13 having a diameter much smaller than the diameter of the anterior wall 8.

The upper larger wall 2 and the lower larger wall 3 include annular toothed anti-expulsion ribs, such as an anterior rib 14, a median rib 15 and a posterior rib 16.

The lateral walls such as the wall 4 may include small lateral openings such as the holes 17 and 18.

As seen in FIGS. 5 and 6 in particular, the width of the implant body, i.e. the distance between the external faces of its lateral walls 4 and 5, is less than the height of the implant, i.e. the distance defined by the external faces of its upper wall 2 and lower wall 3.

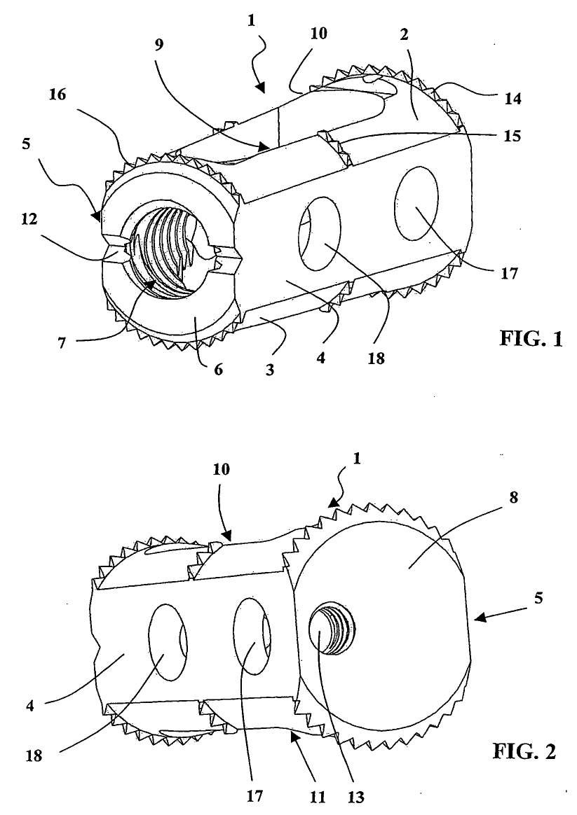

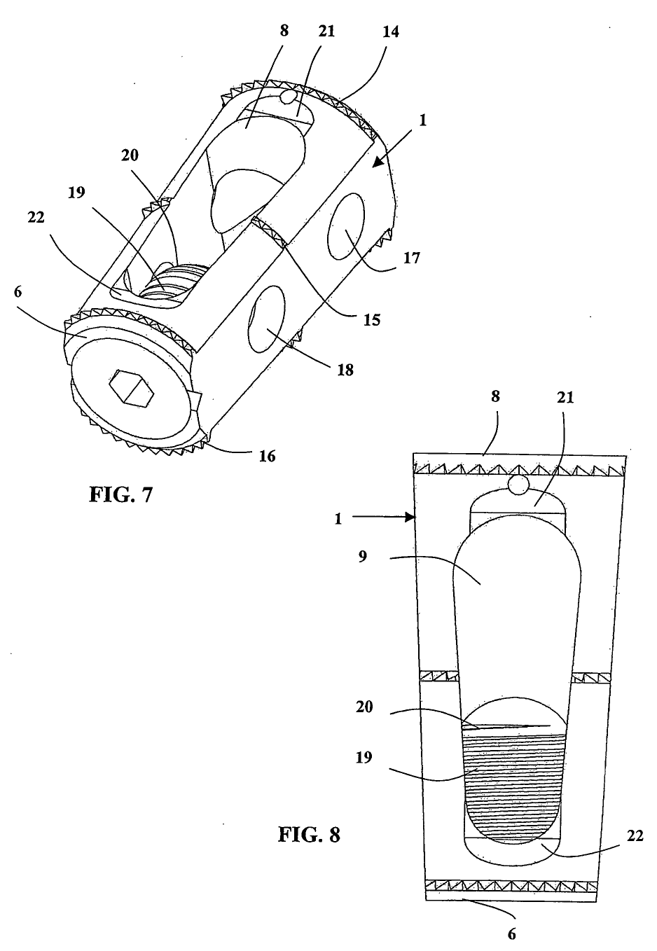

FIGS. 7 and 8, which show the intervertebral implant of the invention in the assembled state, show the implant body 1 as described with reference to FIGS. 1 to 6 and that the implant further includes an interchangeable compression plug 19 fitted by screwing it into the threaded axial hole 7 in the posterior wall 6.

Like the threaded axial hole 7, the interchangeable compression plug 19 has a diameter substantially equal to the smallest width of the interior cavity 9 i.e. the distance between the lateral walls 4 and 5 in the vicinity of the posterior wall 6.

In the embodiment shown in FIGS. 7 and 8, the interchangeable compression plug 19 comprises an interior end portion 20 that is generally conical or ogive-shaped, facilitating its penetration into a graft inserted into the interior cavity 9.

The interchangeable compression plug 19 may be made of titanium, so that it can be detected in an x-ray. On the other hand, the implant body 1 may advantageously be made of a radiotransparent material, advantageously from a PEEK (polyetheretherketone) type polymer.

In this case, a titanium marker may advantageously be provided in the implant body 1 away from the interchangeable compression plug 19.

In the embodiment shown in the figures, the upper wall 2 and the lower wall 3 each have a conical general shape and the upper orifice 10 and the lower orifice 11 are each bordered at its anterior and posterior ends by a respective flat 21 or 22 perpendicular to the lateral walls 4 and 5.

The implant of the invention may be fitted between two adjacent vertebrae to be treated by means of the following steps:

the plate regions between which the implant has to be lodged are squared off,

a bone graft is placed in the interior cavity 9 of the implant body 1,

the implant body 1 is oriented so that its plane lateral faces 4 and 5 are parallel to the plates of the vertebrae to be treated, so that the implant has a minimum height,

the implant body 1 oriented in this manner is inserted between the two adjacent vertebrae to be treated, which are held apart,

the implant body 1 is turned axially 90° to place its upper wall 2 and its lower wall 3 against the plates of the vertebrae to be treated, after which the vertebrae are released,

the interchangeable compression plug 19 is screwed in to compress the graft and thereby to press it against the vertebral plates to be treated.

The present invention is not limited to the embodiments that have been described explicitly, but includes variants and generalizations thereof contained within the scope of the following claims.

1-13. (canceled)

14. Intervertebral implant for stabilizing adjacent vertebrae, comprising a solid biocompatible material implant body having a tubular general structure delimited by an upper wall and a lower wall that are convex and slightly divergent toward the front, two opposite lateral walls that are plane and slightly divergent toward the front, and a posterior wall with a threaded axial hole, with a single interior cavity providing communication between orifices provided in the upper wall and the lower wall, wherein:
the upper wall and the lower wall each comprise a respective single large upper orifice or lower orifice,
an interchangeable compression plug is adapted to be fitted by screwing it into the threaded axial hole in the posterior wall,
the interior cavity is closed toward the front by an anterior wall,
the width of the implant defined by the lateral walls is less than its height defined by the upper wall and the lower wall.

15. Implant according to claim 14, wherein, because of the upper orifice and the lower orifice, the interior cavity is open over the whole of its width between the lateral walls and over the whole of its length between the posterior wall and the anterior wall.

16. Implant according to claim 14, wherein the interchangeable compression plug comprises a conical interior end portion.

17. Implant according to claim 14, wherein the interchangeable compression plug and the threaded axial hole that receives it have a diameter substantially equal to the width of the interior cavity in the vicinity of the posterior wall.

18. Implant according to claim 14, wherein the interchangeable compression plug has a length such that, at the end of screwing it into the threaded axial hole that receives it, its interior end portion penetrates the interior cavity to a distance of at least one quarter of the length of said interior cavity.

19. Implant according to claim 14, wherein the implant has at least two interchangeable compression plugs having different lengths.

20. Implant according to claim 14, wherein the posterior wall of the implant body includes an external diametral groove for actuating axial rotation of the implant.

21. Implant according to claim 14, wherein the anterior wall includes an eccentric threaded hole of smaller diameter.

22. Implant according to claim 14, wherein the upper larger wall and the lower larger wall include annular toothed anti-expulsion ribs.

23. Implant according to claim 14, wherein the interchangeable compression plug is made of titanium.

24. Implant according to claim 14, wherein the implant body is made of a PEEK type polymer.

25. Implant according to claim 24, wherein the implant comprises a titanium marker in the implant body away from the interchangeable compression plug.

26. Implant according to claim 14, wherein the upper wall and lower wall are each of conical general shape and the upper orifice and lower orifice are each bordered at their anterior and posterior ends by a flat perpendicular to the lateral walls.
